(19) United States
(12) Patent Application Publication
(10) Pub. No.: US 2009/0125013 A1
(43) Pub. Date: May 14, 2009

(54) Fat retraction apparatus and method for using same

(76) Inventors: Roza Sypniewski, San Antonio, TX (US); Jozef Sypniewski, San Antonio, TX (US)

Correspondence Address: LOEFFLER JONAS & TUGGEY, LLP, 755 EAST MULBERRY STREET, SUITE 200, SAN ANTONIO, TX, 78212, US

(21) Appl. No.: 12/291,709

(22) Filed: Nov. 13, 2008

Related U.S. Application Data
(60) Provisional application No. 61/002,799, filed on Nov. 13, 2007.

(51) Int. Cl. A61B 18/18

(52) U.S. Cl. 606/33

(57) ABSTRACT

The present invention provides a method and apparatus for non-invasive reduction of excessive fat tissue by externally applying radio frequency (RF) electromagnetic (EM) waves adjusted to specific fat cells absorption frequency and electromagnetic propagation mode. Based on the performed experiments described further, the method and working apparatus has been invented that reduces fat layers without any medication, non-invasively through the intact skin. The invented method and apparatus facilitates the safe fat removal, and may lead to reduction and eradication of obesity and obesity associated diseases.

This application is based upon and claims priority from U.S. Provisional application Ser. No. 61/002,799, which is incorporated herein by reference.

### BACKGROUND OF THE INVENTION

### Background And Field of the Invention

Obesity is a growing problem not only in adults, but also in children. A number of diseases associated with this condition, such as diabetes and/or hypertension, are a large part of the costs of medical treatment and are in the billions of dollars. Despite numerous efforts to date, no treatment has been 100 percent effective in helping these obese individuals. Recent knowledge about the function and role of the adipose tissue in different areas of the body relative to the regulation of the endocrinological system could be used to therapeutic advantage. The reduction of adipose tissue in the abdominal area is very important since those adipocytes contribute to insulin resistance leading to type II diabetes. They also produce estrogen which, in post-menopausal women, can contribute to increased breast cancer risk. Considering the above, it would greatly benefit the health prospects of obese individuals to reduce cellular fat content and possibly the adipocyte number in the abdominal area. The current invention causes fat reduction in selected areas (as needed) of the body through noninvasive (non-surgical procedure) and non-pharmacological therapy. Thus, fat can be reduced noninvasively through intact skin and without damage to other cells and surrounding tissue.

Several patents disclose inventions that relay on focusing energy to select the tissue for removal. As an example, U.S. Pat. No. 5,143,063 proposes to use Barone reflector to focus any energy radiation, including radiofrequency (“RF”) radiation. In the '063 patent, Fellner describes a method for using high electromagnetic energy radiation (“EM”) at the microwave range that may destroy fat cells as temperature rises to 43.3° C.-44.4° C. for 30-40 minutes. It is known to those experienced in the art, that applied microwave energy is not cellular selective and will heat all of the tissue within its sphere of influence. It therefore causes pain and damage to surrounding tissue area.

U.S. Pat. No. 6,607,498 B2 discloses a method and hypothetical apparatus that uses focused ultrasound energy to cause hyperthermia to cells/tissue by focusing energy using direct coupling to the skin through contact sensors.

U.S. Pat. No. 5,769,879 illustrates a relay on phase array transducer where a multiplicity of transducers are controlled by an adjustable phase-shifter. The wavefront of radiated energy is adjusted, so assuming that the structure of EM properties of irradiated tissue is known, the wavefront is focused at the point of a designated area.

The major problem with these methods is that most of employed transducers use a near-field propagation mode with strong interaction between their properties and random EM properties of irradiated tissue. Also, the radiated wavefront from these transducers passes through random and highly dispersive media before reaching the desired region. As it is seen in other state of the art areas (RADAR, medical imaging) this problem is very difficult or impossible to solve. The above mentioned patents do not address this problem.

Several patents, represented by U.S. Pat. No. 6,461,350 B1 and 6,719,754 B2, disclose that tissue selection is achieved by direct electrical contact with fat cells selected for removal. Thus, use of these prior inventions require invasive techniques in order to employ them.

Likewise U.S. Pat. No. 7,217.265 B2 discloses direct Infrared Radiation (IR) method to select tissue chosen for removal. In this invention the direct exposure of fat tissue is required that means that incisions and cuts through the skin are needed to physically reach fat layer under the skin by the beam of IR lasers.

In U.S. Pat. No. 5,507,790 issued to Weiss, a method is described in which medication is applied that allows focusing of the applied electromagnetic energy to the skin over the desired area of the underlying fat tissue to increase fat lysis.

The invention disclosed in U.S. Pat. No. 7,250,047 B2 requires vacuum to be applied to the skin to articulate fat tissue for through propagation EM exposure.

### SUMMARY OF THE INVENTION

Electromagnetic radiation consists of waves of electric and magnetic energy moving together. Often the term electromagnetic field or radiofrequency (“RF”) field may be used to indicate the presence of electromagnetic or RF energy. An RF field has both an electric and a magnetic component (electric field and magnetic field). RF waves can be characterized by a wavelength and a frequency. The wavelength is the distance covered by one complete cycle of the electromagnetic wave, while the frequency is the number of electromagnetic waves passing a given point in one second. Biological effects that result from heating of tissue by RF energy have long been observed. High levels of RF radiation has the ability to rapidly heat biological tissue. Tissue damage may result due to the body's inability to dissipate the heat generated.

The current invention utilizes a specific interaction between irradiated tissue and a transducer to adjust the transducer properties to match EM properties of designated fat cells. Further, the current invention uses non-invasive EM radiation to induce fat cell apoptosis, necrosis and oncotic death. The EM transducer of the present invention has no direct electrical contact with the tissue. The present invention allows the transducer to be used non-invasively from the target tissue by dielectric layer. Consequently, the tissue selection is achieved by adaptive matching of fat EM property with mode and frequency of emitted EM field from the transducer included in the apparatus of our invention.

Fat cells have unique electrical and magnetic properties as compared to other tissues. Most tissue has an electrical constant of approximately 80, while adipose tissue has a very different electrical constant of approximately 13. The impedance of the cell is the ratio of the electrical constant to the magnetic constant. The present invention makes use of near field radiation. It uses primarily an electric field. The user can adjust the polarity of the field, and the frequency. The radiation does not need to induce heat so as to burn the tissue it is in contact with, but by adjusting the polarity and frequency to match the impedance of the adipose cells, the optimized radiation causes an affectation of the fat cells. The affectation may be to heat the cells from the inside out so as to cause reduction in the lipid globules found within the cells, or to the point of cell membrane damage or cell death. The fat cells are heated so as to cause a cell affectation, but the skin and other tissue are not (although the other tissue may be warmed).

In one embodiment of the present invention, a method is provided for lipid depletion within the adipocytes without cell death or damage by the application of optimized RF energy. By adjusting the components of the RF energy application, such as application time and power, the method may be adjusted so as to induce the death of fat cells within the adipose tissue through mechanisms of lysis, apoptosis, necrosis or oncotic death.

The present invention could also be used to eliminate tumors of adipose tissue, benign lipomas, and malignant liposarcomas originating from unilocular adipocytes and in treatment of hibernomas that originate from multilocular adipose cells. It may further be used in the reduction of breast size by elimination of excessive fat cells within the mammary tissue. Generally, excess fat cells in any area of the body can be killed and removed.

The present invention may be modified to reduce fat globules within the adipose cells rather than killing the cells themselves. Each fat cell has a cell wall, and inside is the fat, stored as tiny liquid droplets of lipid. Like other kinds of cells, fat cells have a cell wall and a nucleus. Unlike other kinds of cells, however, globules of liquid fat push the nucleus of a fat cell over to the side of the cell. Almost all of the fat cell's interior space (generally more than 90%) is taken up by the liquid lipid globules. As more fat is stored in the cell, the globules get larger or the number of globules may be increased. The cell can eventually grow to many times its original size. The user of the present invention can choose to reduce the amount of fat globules within the fat cells by modifying the radiation frequency and polarity from what is optimum for cell death to a frequency and polarity that causes globule reduction within the cells.

The present invention, an apparatus is described for use of the fat removal (depletion) from the adipocytes without inducing damage or death of adipocytes, but also can be used in activating a death signaling mechanism through apoptosis or necrosis for activation of natural-self removal of adipose tissue underlying the skin or in any area of the body of the subject. A three dimensional (“3D”) multi-mode type electromagnetic RF transducer, or transducers, is applied to the subject's skin; and an electrical RF generator, having a controller and waveform optimizer therein, energizes the RF transducer to generate and to transmit through the subject's skin non-invasively without any incisions or punctures, electromagnetic waves activating selectively the death signaling pathway inside the fat cells of the selected area of the adipose tissue without damaging non-adipose tissue. In addition to an RF frequency generator, the present invention may incorporate a controller that permits the invention to deliver a RF wave inducing death signal to fat cells that have different EM properties from surrounding subcutaneous cells. The RF generator and controller targets high wave impedance isotropic cells. Further features and advantages of the invention will be apparent from the description below.

### Identification Of The Fat Regression Parameters And Measuring Methods

### Adipose Tissue Preparation:

The skin with underlying fat tissue and muscles were dissected of a male hog, weighing approximately 150 lb. The dissection was performed immediately after the animal sacrifice. The selected block of tissue (11 cm×8 cm) was cut from the top of the skin into the underlying tissue to match the prototype transducer unit of RF exposure which is generally from the top of the skin 4-5 cm deep into the fat and muscles tissue. The dissected tissue was kept at 4° C. until used, but no longer than 12 hr from the sacrifice.

EM Radiation with Adaptive Mode (ERAM) in which flex quadrature rotate RF waves was applied, followed by instant excision of a portion of tissue 1 cm×1 cm×4 cm that contained skin, connective tissue, fat and muscles. The specimen was preserved in histological grade, 10% buffered formaldehyde for more than 48 hours. The preservation was followed by standard histological tissue preparation—embedding, sectioning and staining the tissue with Hematoxylin and Eosin and Oil Red O Stain. The tissue was processed and sections were cut longitudinally in order to allow the evaluation of the effect of adaptive mode EM radiation on the fat and surrounding tissue. The tissue was stained and a pathology evaluation was conducted.

### Microscopic Analysis:

Microscopic analysis was performed using light microscopy, at ×50, ×100, and ×400 magnification. A video camera, with PCP-based image capturing software, connected to the microscope was used to record the pictures. Five fields in each slide were examined at each magnification. Tissue damage or effect could be indicated by ghost cell appearance, membrane disruption, reduction in cell size or content (as demonstrated by techniques such as fat specific staining), and cyst formation, varying in size and extent. A magnification of ×400 was used to examine fine details of potential pathology in the field of view. This analysis was performed in a “blind manner” independently by a scientist and certified pathologist. Fat reduction in the adipocytes was microscopically demonstrated with the assistance of fat specific staining (such as “oil red O staining”).

Two scales were defined for the evaluation of the effects on (1) fat cells and (2) non-fat cells:

(1) Fat tissue score (grade 0-4):
Grade 0—no fat reduction or damage, intact undamaged tissue with normal appearance (up to 10% damage might be technical and therefore was counted as grade 0).
2. Grade 1—minimal change or damage to fat cells, 11-25% lipid disappearance in fat cells, no visible change to membrane or cell appearance.
3. Grade 2—visible fat depletion in adipocytes, 26-50% lipid disappearance in fat cells, no detectable changes to adipocyte integrity, membranes, organelles, or appearance.
4. Grade 3—substantial fat reduction in adipocytes, 51-75% lipid disappearance in fat cells, no or minimal changes to adipocyte integrity, membranes, organelles, or appearance.
5. Grade 4—massive to total fat reduction in adipocytes, 76-100% lipid disappearance in fat cells, scattered disruption of adipocyte membranes, some enlarged adipocytes, and changes to adipocyte integrity, membranes, organelles, or appearance.

(2) Non-fat tissue score:
1. Grade 0—no damage: intact, undamaged tissue
2. Grade 1—damage: description of the damage will be added if any damage will be observed. Potential damage to other cells of the adipose tissue (connective, peripheral nerves, blood vessels) and skin and muscle was evaluated at all microscopic magnifications ×50, ×100, and ×400 to uncover even smallest alterations of exposed tissue.

### Results and Conclusions:

In order to determine and optimize a therapeutic range of RF frequencies, the optimal EM radiation mode has to be defined. Adaptive adjustment of EM radiation is necessary to selectively target adipose tissue. The disclosed method reduced lipid content in the fat cells.

### BRIEF DESCRIPTION OF THE DRAWINGS

The invention is herein described, by way of example only, with references to the accompanying drawings, wherein:

FIG. 1. is a block diagram that illustrates principal of operation of apparatus constructed in accordance with the present invention.

FIG. 2 is a circuit diagram illustrating one form of apparatus constructed in accordance with the present invention.

FIG. 3a and 3b are perspective and cut-away views, respectively, of the RF transducer of the present invention.

FIG. 4a and 4b are diagrams illustrating, respectively, a form of RF quadrature wave transducer and transverse wave transducer, used in conjunction with the present invention.

FIG. 5 illustrates basic interaction between RF transducer constructed in accordance with the present invention and tissue structure.

FIG. 6 is a flowchart that illustrates operation of program that controls operation of an apparatus constructed in accordance with the present invention.

### DETAILED DESCRIPTION OF THE PREFERRED EMBODIMENT

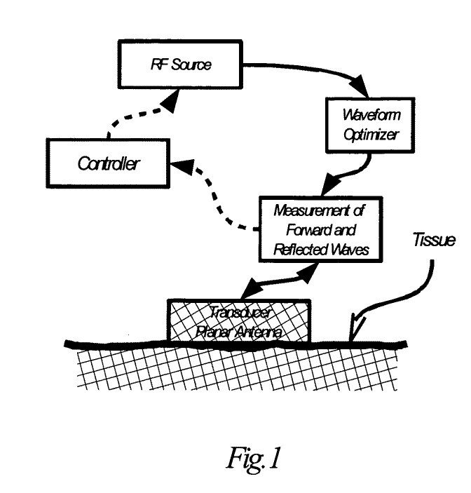

Referring to the figures, FIG. 1 illustrates an embodiment of the present invention. It shows that apparatus will contain at least one EM RF transducer that is applied to the subject's skin to transmit there through EM RF wave with computer controlled and adjustable frequency. The polarity of the emitted RF field is also computer controlled and matched to the EM properties of irradiated fat tissue. Energy of this radiation targets mainly the adipose tissue: electrically actuating the EM RF transducer or transducers. The RF field is of sufficient intensity to cause selective death of said adipose tissue without damaging, or with only minimal damage to, surrounding and intercalated cells of non-adipose tissue type.

An electromagnetic transducer may be slid over the skin to destroy the adipocytes by causing absorption into cells to the point that the cells swell and lyse, damaging the adipocyte membranes after the time of the exposure. Selectivity adjusted RF radiation targets the adipocytes containing fat molecules only, and does not significantly affect other surrounding cells, such as skin, connective tissue, blood vessels, nerves, muscle fibers and bone. Also, the procedure can be carried out in any office setting without the need for an operating room and anesthesia in a hospital.

Further features in the preferred embodiments of the invention include, without limitation:
(1) The electrical peak output power is preferably from 1 mW to 1 kW, more preferably adjustable and computer controlled from 0.1 W to 1 W for tissue identification. And 10 W to 200 W for actual fat tissue irradiation.
(2) Frequency is adjustable and computer controlled from DC to 2 GHz, more preferably from 1 MHz to 50 MHz.
(3) Radiation polarity is adjustable in full 3D degree of freedom and computer controlled.
(4) Apparatus is capable of generating EM radiation in circular and linear polarity.
(5) Both forward and reflected power of emitted radiation is monitored to determine, in real time, the electrical properties of irradiated tissue.

In contrary to present state of the art which attempts to utilize wavefront formation (focusing), the present invention targets disposable fat tissue by matching emitted EM radiation to EM properties of targeted fat tissue. The EM properties of fat tissue significantly differ from equivalent properties of surrounding area (muscle, blood vessel, epidermal). For example, the dielectric constant of fat tissue for 50 MHz is from 85 to 97, while the same constant for muscle tissue is from 11 to 13 (Schwan H P, Electrical Properties of Tissues and Cell Suspension; Adv. Bio. Med. Phys 5:147-209, 1957).

Other EM properties of nonfat tissue differ significantly from its fat tissue equivalent as well. Blood vessel and muscle cells are highly anisotropic for lower range of RF radiation. In contrast, fat tissue is almost perfectly isotropic.

The present invention emits EM radiation with two degrees of freedom. The first degree of freedom is the relative position of the electric and magnetic components of EM radiation—EM propagation mode. The second degree of freedom is the frequency of emitted EM radiation. Both frequency and EM propagation mode can be individually adjusted and computer controlled. To monitor which area is targeted by emitted radiation, the present invention utilizes interaction between EM transducer and irradiated tissue. It monitors both radiated and reflected power of transmitted EM radiation. It further optimizes EM radiation to target isotropic tissue with a high value of complex wave impedance.

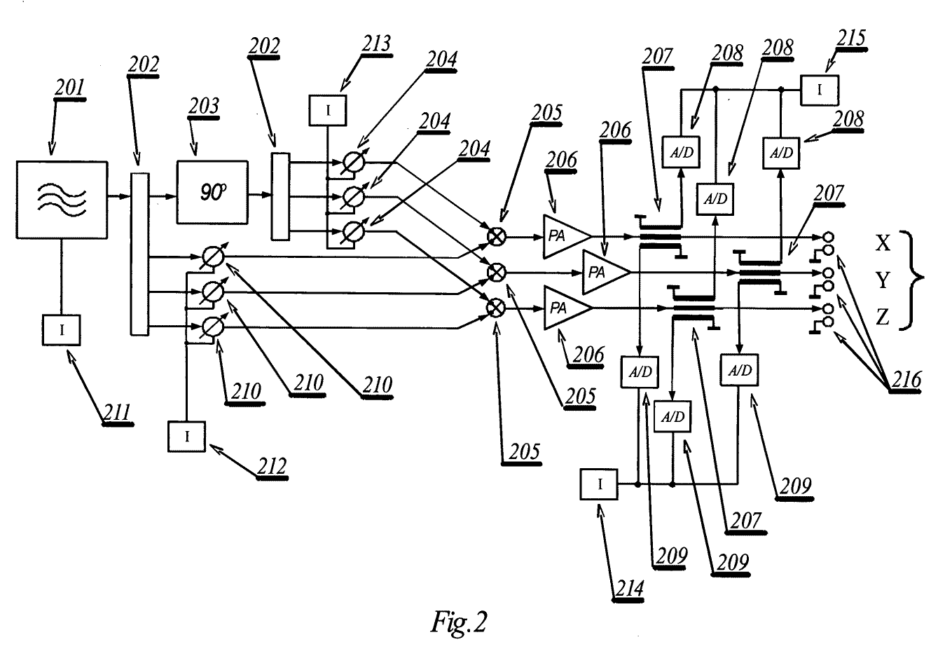

FIG. 2 is a block diagram of one embodiment of the apparatus of the present invention. The main signal is generated at the digitally controlled oscillator 201. The frequency of the oscillator 201 is computer controlled through first computer interface 211. The signal from the oscillator 201 is split at a first splitter 202a and sent to digitally controlled, second attenuators 210. These computer controlled second attenuators 210 can adjust the ratio of the signals IX, IY, IZ. The same signal passes through a broadband Hilbert transform filter 203 and output of that filter 203 is connected to a second splitter 202, and then to a set of digitally controlled first attenuators 204. The signals from combiners 205 are amplified via the power amplifiers 206 and through the pairs of directional couplers 207 sent to the connectors 216 and to the transducer (not shown). The quadrature signals from the first attenuators 204 and second attenuators 210 are combined in combiners 205 forming Xₒ, Yₒ, Zₒ at the desired polarization mode at the transducer. Couplers 207 combined with standard A/D converters 208 and 209 measure both power irradiated and reflected from the tissue. Data is acquired by main computer using fourth computer interface 214 and fifth computer interface 215. Also, through second and third computer interfaces 212 and 213, the computer can control any radiation mode of the EM radiation by changing phase and amplitude of the signal X, Y, Z assigned to each transducer coordinate.

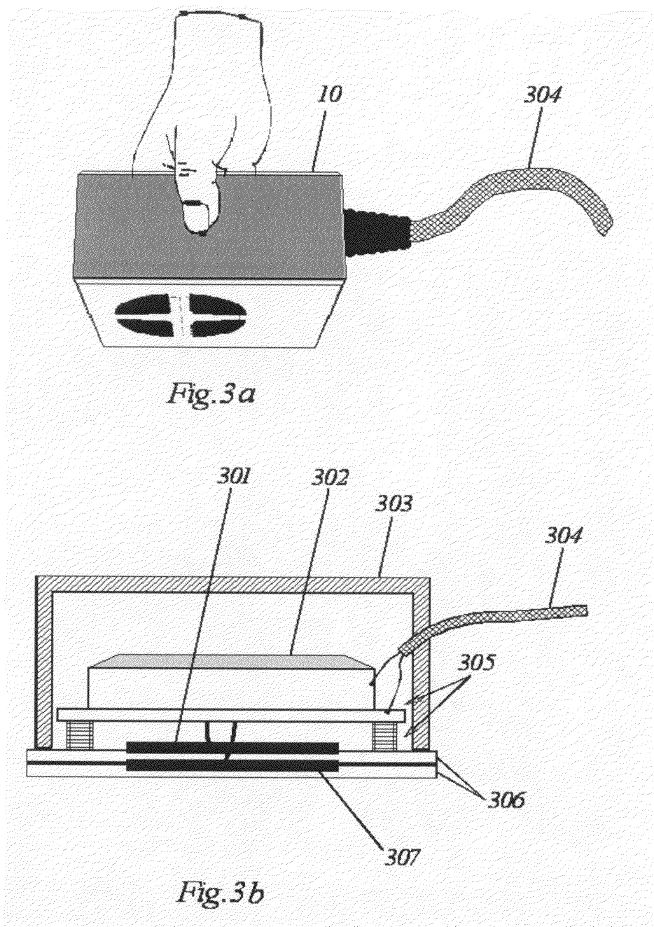

FIG. 3a shows a perspective view of the transducer 10. The signal from the electronic circuit presented in FIG. 2 is connected to transducer 10 via the cable 304. An apparatus to irradiate a target area of fat cells for the non-invasive affectation of the fat cells could be constructed in a number of embodiments, but the embodiments would share a RF energy generator and a transmitter such that the apparatus would allow for adjusting the properties of the RF energy.

FIG. 3b is a cut-away of the transducer 10. Using cable 304, a signal is sent to array of matching circuits 302 and then to a first planar antenna 301 and a second planar antenna 307. Second planar antenna 307 is fed by the signals Xₒ and Yₒ.

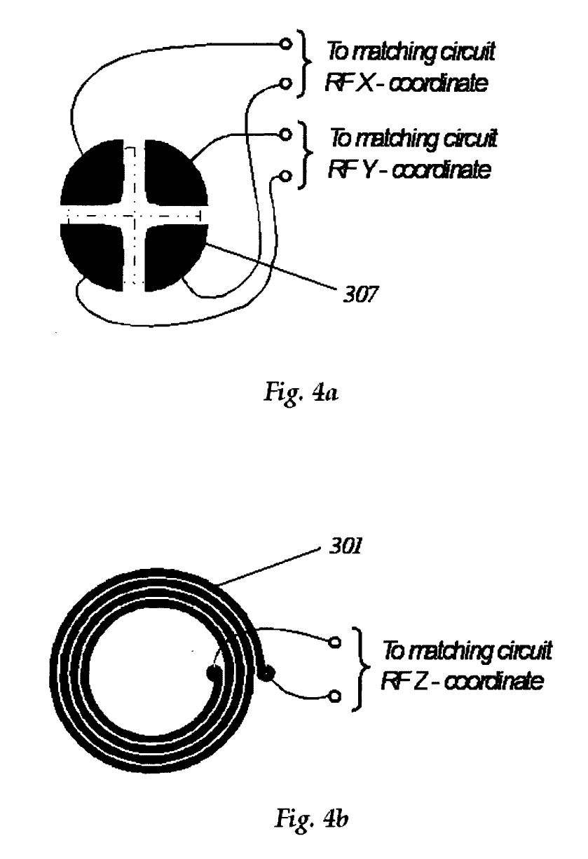

FIG. 4a shows example shape of radiation electrodes of antenna 307. This antenna generates linearly polarized Transverse EM (TEM) waves at any polarization angle. Adjusting the ratio of IX and IY signals from FIG. 2 one can change desired polarization angle of TEM radiation. Also, the antenna 307 can generate circularly polarized TEM waves. Mixing the signals IX, IY and QX, QY respectively the phase of the signals X and Y can be shifted simulating electrical field rotation of EM radiation. Signal Zₒ supplies planar antenna 301. This antenna transmits EM radiation at the Normal EM (NEM) mode.

FIG. 4b shows that the antenna 301 forms a coil and generates magnetic component of EM radiation. Adjusting signals IX, IY, IZ and QX, QY, QZ one can steer an EM radiation vector at any direction emulating linear or circular polarized waves. The working components of the transducer 10 and its mounting hardware 305 are encapsulated in an enclosure 303.

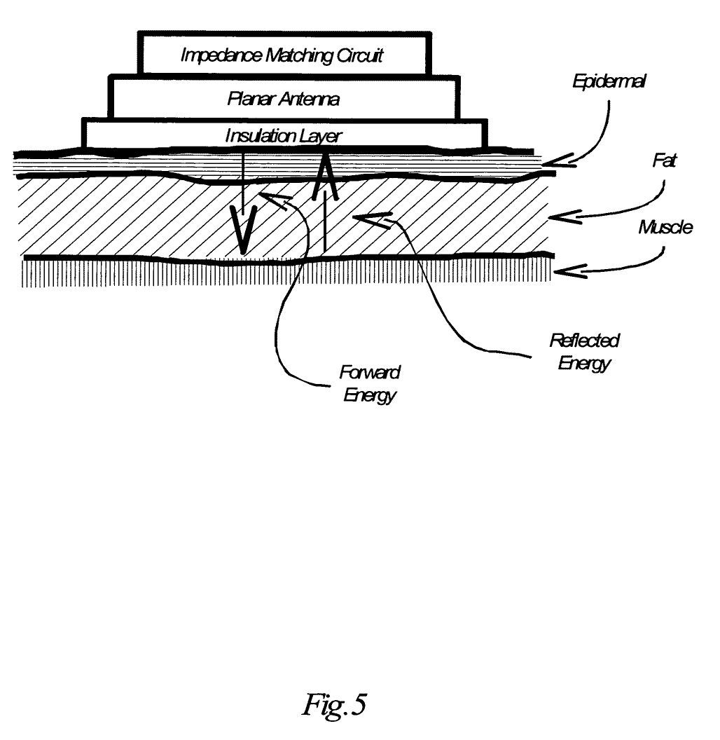

FIG. 5 shows typical layers structure of epidermal, fat and muscle tissue. The RF planar antennas shown in FIG. 3 and FIG. 4 work in near field propagation mode with strong interaction between RF transducer and irradiated tissue. Initial radiated RF energy is reflected mainly at the wave impedance discontinuity that is at the boundary of tissue layer with different values of complex permittivity. The reflected energy is collected back by the planar antennas and transferred to the circuit illustrated in FIG. 2. The directional couplers 207 (FIG. 2) acquire the signal from antennas and transfer it to a computer via computer interfaces 214 and 215. The intensity of reflected energy is highly dependent upon frequency and radiation, and it is defined by the dielectric constant distribution of the tissue surrounding RF transducer.

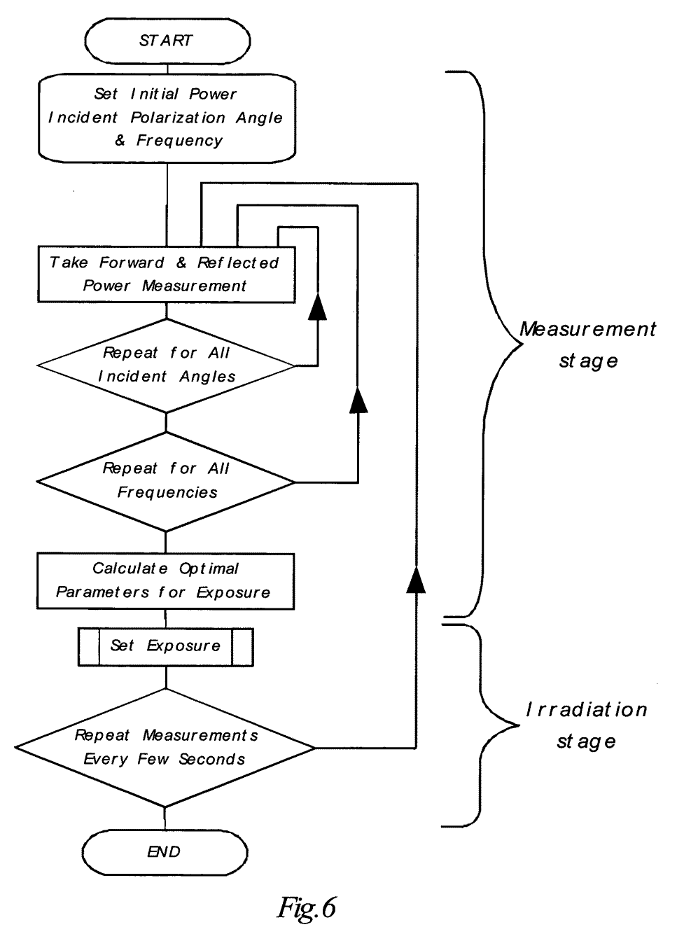

FIG. 6 shows flowchart of the method of the present invention. It illustrates that the RF irradiation process is divided into two stages; a measurement stage and an irradiation stage.

At the measurement stage, RF power is set to allow adequate measurement but bellow the threshold of any significant tissue reaction. During this stage the interaction between transducer and tissue is evaluated as described earlier. The multitude of reflected power measurement is taken for different polarization characteristics and for various frequencies. This series of measurements are collected where single measurement constitutes projection of the dielectric constant distribution of the surrounding tissue. The relevant inverse projection algorithm is applied on collected data to determine the surrounding tissue structure. Also, based on collected data optimal polarization mode and frequency is calculated, so irradiated RF energy will affect mainly adipose tissue.

At least one Radio Frequency (RF) emitter is applied to the subject's skin to deliver RF waves which are absorbed mainly by adipose tissue. A low level of radiation is applied and monitored to see how much radiation is absorbed by the body. The field may be rotated in order to measure the absorption. A tissue scan may be used to measure the impedance of tissue directly surrounding the sensor. From the measured amount of absorption the amount and thickness of the fat tissue may be determined. From this, the optimum radiation frequency and polarity may be calculated. The optimized RF energy matches the wave impedance of the target fat tissue. The emitter is adjusted so that the RF radiation selectively targets and triggers cell death using either a known, or calculated, optimum radiation frequency and polarity, and the tissue is irradiated. Fat tends to be a good insulator and as such, tends to hold the applied heat. The applied radiation selectively heats the fat cells causing cell death or reduction of the lipid mass within the fat cells. Consequently, there is a release of stored fat from adipose cells into surrounding unaffected tissue (skin, blood vessels, nerves, muscles and bones). A near field RF energy quadrature patch antenna may be applied to transfer RF energy into said target area of said fat cells. In determining the optimized RF energy, the quadrature ratio of said RF energy may be adjusted and the optimum linear polarization angle modified. Further, the quadrature signal may be adjusted so as to produce a circular radiation pattern, and the quadrature signal circular radiation pattern may be at an arbitrary plain angle. A near field RF radiation loop antenna may be applied in order to generate a magnetic component of Electromagnetic Radiation in near field RF radiation region to produce a transverse RF polarization. In optimizing the RF energy, the distribution of complex permittivity in tissue surrounding said near field RF radiation loop antenna may be measured. Further in optimizing the RF energy, backscatter of RF energy may be measured.

At the irradiation stage, the optimized polarization, frequency and RF power is used to irradiate the target tissue for a predetermined period of time. This time period lasts until the target fat cells are affected, where affected means one of a reduction of the lipids within the cell, disruption of adipocyte membranes or lysing, enlarged adipocytes, or changes to adipocyte integrity, membranes, organelles, or appearance.

Also, during this stage the interaction between tissue and transducer is measured to monitor any changes during irradiation. The entire procedure is repeated as necessary to complete an irradiation process.

Although the invention has been described with reference to specific embodiments, this description is not meant to be construed in a limiting sense. Various modifications of the disclosed embodiments, as well as alternative embodiments of the inventions will become apparent to persons skilled in the art upon the reference to the description of the invention. It is, therefore, contemplated that the appended claims will cover such modifications that fall within the scope of the invention.

I claim:

1. A method for the non-invasive affectation of fat cells from a target area of tissue comprising irradiating said target area with an adjustable radiofrequency (“RF”) energy, wherein as a result, a portion of said fat cells suffer at least one of: reduction of the intracellular lipids, membranes disruption or lysing, enlargement, or changes to their integrity, membranes, organelles, or appearance.

2. A method for the non-invasive affectation of fat cells from a body comprising:
identifying a target area of said fat cells;
optimizing the properties of radiofrequency (“RF”) energy so as to match the wave impedance of said fat tissue; and
irradiating said target area with said optimized RF energy until fat cells are affected.

3. The method of claim 2, further comprising applying a near field RF energy quadrature patch antenna to transfer RF energy into said target area of said fat cells.

4. The method of claim 3, further comprising:
adjusting the quadrature ratio of said RF energy; and
modifying the optimum linear polarization angle.

5. The method of claim 3, wherein said quadrature signal may be adjusted so as to produce a circular radiation pattern

6. The method of claim 3, wherein said quadrature signal circular radiation pattern at arbitrary plain angle

7. The method of claim 2, further comprising:
applying near field RF radiation loop antenna to generate a magnetic component of Electromagnetic Radiation in near field RF radiation region producing transverse RF polarization.

8. The method of claim 2, wherein in said irradiating step, affected means one of: a portion of said fat cells suffer at least one of: reduction of the intracellular lipids, membranes disruption or lysing, enlargement, or changes to their integrity, membranes, organelles, or appearance.

9. The method of claim 2, wherein said optimizing step further comprises measuring the distribution of complex permittivity in tissue surrounding said near field RF radiation loop antenna.

10. The method of claim 2, wherein said optimizing step further comprises measuring backscatter of RF energy.

11. An apparatus for the non-invasive affectation of fat cells from a body comprising:
a radiofrequency (“RF”) energy generator;
a transmitter; and
wherein said apparatus provides for optimizing the properties of RF energy so as to match the wave impedance of said fat tissue; and irradiating said target area with said optimized RF energy until fat cells are affected.

12. The apparatus of claim 11, further comprising:
a controller;
a transducer;
a monitoring device for depth and directionality of said RF energy.
and complete integration system of fat reduction area selection with real time monitoring of actual treatment, and adjustment capability to facilitate RF desirable emission to penetrate or to transfer through the skin, superficial tissue (human or animal) to treat shallow or deeper layers of fat in one RF treatment apparatus.

13. The apparatus of claim 11, wherein said apparatus provides means for adjusting the quadrature ratio of said RF energy and modifying the optimum linear polarization angle.

14. The apparatus of claim 13, wherein said quadrature signal may be adjusted so as to produce a circular radiation pattern.

15. The apparatus of claim 11, further comprising a directional coupler circuit on the RF supplying line, wherein said directional coupler circuit measures backscatter of RF energy in and an inverse algorithm to backscatter RF radiation to measure a complex permittivity distribution in tissue surrounding said antenna.
